(19) United States
(12) Patent Application Publication
(10) Pub. No.: US 2020/0069832 A1
(43) Pub. Date: Mar. 5, 2020

(54) FRAGRANCE RELEASING DEVICE

(71) Applicant: TAKASAGO INTERNATIONAL CORPORATION, Tokyo (JP)

(72) Inventors: Thomas F. Santini, Doylestown, PA (US); Anthony R. Budraitis, Lambertville, NJ (US); Alpa Shah, Morris Plains, NJ (US); Angelique Nadau, Westwood, NJ (US)

(73) Assignee: TAKASAGO INTERNATIONAL CORPORATION, Tokyo (JP)

(21) Appl. No.: 16/610,618

(22) PCT Filed: May 8, 2018
(86) PCT No.: PCT/US2018/031707

§ 371 Date: Nov. 4, 2019

Related U.S. Application Data
(60) Provisional application No. 62/503,312, filed on May 8, 2017.

(51) Int. Cl. A61L 9/12

(52) U.S. Cl. CPC A61L 9/12; A61L 2209/131

(57) ABSTRACT

A fragrance delivery device, comprising a vapor releasing oleophobic microporous layer; a flexible impermeable barrier layer coupled to the vapor releasing oleophobic microporous layer at a perimeter thereof, wherein the microporous layer and barrier layer define a cavity; and a reservoir substance disposed in the cavity, wherein a volatile material of the reservoir substance is releasable from the fragrance delivery device via the micropores of the microporous layer when the fragrance delivery device is at ambient temperature.

### CROSS-REFERENCE TO RELATED APPLICATIONS

The present application claims priority to U.S. Provisional Patent Application Ser. No. 62/503,312, filed on May 8, 2017, the content of which is incorporated herein by reference in its entirety.

### FIELD

The present disclosure relates to a fragrance releasing device having a microporous membrane that is ready-to-use without further energizing the fragrance containing medium therein.

### BACKGROUND

A variety of air freshening forms exist in the marketplace offering the consumer an array of performance options. Such air freshening forms include energy-driven units, absorbent pad devices, and traditional membrane devices. Energy driven or power-assisted units, whether they are based on the use of heat, a fan, or both to assist in fragrance dispersion, have been proposed. The energy assisted fragrance dispersion can contribute to a greater fragrance awareness. However, such units require plugging them into the wall or batteries and can increase costs that makes these units expensive to manufacture and operate. These devices are also often difficult to transport and cumbersome for on-the-go users.

A traditional fragrance carrier is described in U.S. Pat. No. 7,926,735 to Mobley et al. In Mobley, the carrier substrate is a paperboard card, which is impregnated with a fragrance solution for diffusion. However, when fragrance is absorbed directly onto a carrier such as paperboard or paper-like materials, the fragrance release is inordinately high when the carrier is newly exposed and subsequently drops dramatically as time passes. Such carriers require additional housing material to surround the carrier so that the fragrance does not transfer onto a user's hands or personal effects. Such carriers can also overpower a small space, such as a locker, and can create an unpleasant experience. Furthermore, such carriers are limited in the kinds of fragrances that can be used.

Traditional membrane devices have become appreciated for their lightweight construction and generally smaller size. Typically, such devices are constructed with a release membrane covering the full expanse of a shallow tray, which contains the fluid contents to be dispensed. Such devices, however, are very limited in the kinds of fragrances that can be used as only certain vapor pressured fragrances can emanate from such devices. These devices often fail and the fragrance mediums therein can seep through the traditional membrane to cause unpleasant conditions.

Therefore, there remains a need to provide a fragrance-releasing device, which provides the benefits of the above-mentioned devices, while improving upon at least the deficiencies. More particularly, it is desirable to have a fragrance-releasing device that can be more safely used near children and pets, provides for more efficient clean-up after use, enables utilization of a broad range of fragrance types, provide reduced seepage or leakage of the fragrance medium, is cost effective and easily transportable, and can provide an optimal fragrance experience over the course of its use. The present disclosure addresses these and other needs in further detail below.

### SUMMARY

The purpose and advantages of the disclosed subject matter will be set forth in and are apparent from the description that follows, as well as will be learned by practice of the disclosed subject matter. Additional advantages of the disclosed subject matter will be realized and attained by the devices particularly pointed out in the written description and claims hereof, as well as from the appended drawings.

The present disclosure relates to a fragrance releasing device having a vapor-releasing microporous layer and reservoir substance and methods of using such devices. The fragrance releasing device disclosed herein advantageously provides an optimal fragrance experience while avoiding the negative drawbacks associated with traditional devices.

The present disclosure also provides for the fabrication of different sized devices such that two or more devices of different fragrance types could be used at the same time to offer the user creative control over their indoor fragrance experience. Such devices can be smaller or larger in size.

In addition, the use of a fully-sealed and self-contained device as presently disclosed also permits the device to be used more globally in locations without the need of a warming or energizing unit. The device can be oriented in any configuration, such as adhered to the back of a locker, and is not limited to a horizontal orientation often required by conventional devices.

To achieve these and other advantages and in accordance with the purpose of the disclosed subject matter, as embodied and broadly described, the disclosed subject matter includes a fragrance delivery device. The fragrance delivery device has a vapor releasing microporous layer having a plurality of micropores therein. The device further has a flexible impermeable barrier layer coupled to the vapor releasing microporous layer at a perimeter thereof, wherein the microporous layer and barrier layer define a cavity. A reservoir substance is disposed in the cavity, wherein a volatile material of the reservoir substance is releasable from the fragrance delivery device via the plurality of micropores when the fragrance delivery device is exposed to an ambient temperature.

As further embodied herein, the vapor releasing microporous layer of the device can be a vapor releasing oleophobic microporous layer.

In accordance with another embodiment of the disclosed subject matter, a method of manufacturing a fragrance delivery device is disclosed. The method includes providing a vapor releasing microporous layer and coupling a flexible impermeable barrier layer to the vapor releasing microporous layer such that microporous layer and barrier layer define a cavity. The method further includes depositing a reservoir substance into the cavity, wherein a volatile material of the reservoir substance is releasable from the fragrance delivery device via micropores of the microporous layer when the fragrance delivery device is at ambient temperature, and sealing the vapor releasing microporous layer with the flexible impermeable barrier layer at a perimeter thereof.

### BRIEF DESCRIPTION OF THE DRAWINGS

FIG. 1 depicts an exterior perspective view of a filled, sealed device in accordance with an embodiment of the disclosed subject matter. The device includes a name printed on the available printed surface.

FIG. 2A depicts a cross-sectional perspective view of a device in accordance with an embodiment of the disclosed subject matter.

FIG. 2B depicts an exploded view of the components of the device of FIG. 2A, according to the disclosed subject matter.

FIG. 3 depicts a cross sectional perspective view of a device in accordance with another embodiment of the disclosed subject matter.

FIG. 4 depicts an exterior perspective view of a delivery system with a device and a display unit in accordance with an embodiment of the disclosed subject matter.

FIG. 5 depicts the transmission of the top, mid, and base notes of a volatile material through a vapor releasing membrane (shown in limited cross sectional view) according to the disclosed subject matter.

FIG. 6 depicts the transmission of the top, mid, and base notes of a volatile material through a traditional prior art membrane (shown in limited cross sectional view).

FIG. 7 depicts a cross-sectional perspective view of two reduced-size devices, according to the disclosed subject matter.

FIG. 8 depicts a cross-sectional perspective view of the device of FIG. 2A without the reservoir substance, according to the disclosed subject matter.

FIG. 9A depicts a plurality of reduced size devices on a display tray, according to the disclosed subject matter.

FIG. 9B depicts a plurality of reduced size devices on a display tray with a containment lid, according to the disclosed subject matter.

### DETAILED DESCRIPTION

### Definitions

The terms used in this specification generally have their ordinary meanings in the art, within the context of this disclosure and in the specific context where each term is used. Certain terms are discussed below, or elsewhere in the specification, to provide additional guidance to a person of ordinary skill in the art describing the compositions and methods of the disclosure and how to make and use them.

As used herein, the use of the word “a” or “an” when used in conjunction with the term “comprising” in the claims and/or the specification can mean “one,” but it is also consistent with the meaning of “one or more,” “at least one,” a plurality, and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “microporous” means average pore sizes not exceeding about 1 μm or micrometer. As such, a microporous membrane means a membrane having pores with an average pore size that does not exceed about 1 μm.

As used herein, the term “oleophobic” means a membrane material capable of preventing the passage of oil and is reported by an oil repellency grade, as described in more detail below.

As used herein, the term “porosity” is used to indicate the void fraction or “empty” space of a membrane material. This is a fraction of the volume of voids over the total volume listed as a percentage between 0% and 100%.

As used herein, the term “permeability” is a measurement of air passage through a membrane material and may be, for example, measured according to ASTM D726-58, Method A, reported in Gurley seconds/50 cc.

### Fragrance Releasing Device

Reference will now be made in detail to various embodiments of the disclosed subject matter, non-limiting examples of which are illustrated in the accompanying drawings. The device presented generally is intended for releasing a volatile active material, such as a fragrance or fragrance composition, into the surrounding environment.

As embodied herein, the device includes a “reservoir” defined by at least one vapor releasing microporous membrane. In certain embodiments one or more vapor releasing microporous membranes can be used to form a cavity or reservoir therebetween. In certain embodiments, the device can further include an impermeable barrier layer, and a cavity or reservoir can be formed between the impermeable barrier layer and the one or more vapor releasing microporous membranes. In certain embodiments, the one or more vapor releasing microporous membranes can be vapor releasing oleophobic microporous membranes. The reservoir includes at least one volatile active material. In certain embodiments, the volatile active material is a non-thickened fragrance oil. In other embodiments, the volatile active material is a thickened, i.e., more viscous, fragrance solution. In certain yet other embodiments, the reservoir includes a gel-based matrix and the volatile active material, i.e., fragrance, is contained within the matrix.

As used herein, the “volatile active material” or “volatile material” refers to a volatile fragrance compound or fragrance composition containing one or more volatile fragrance compounds.

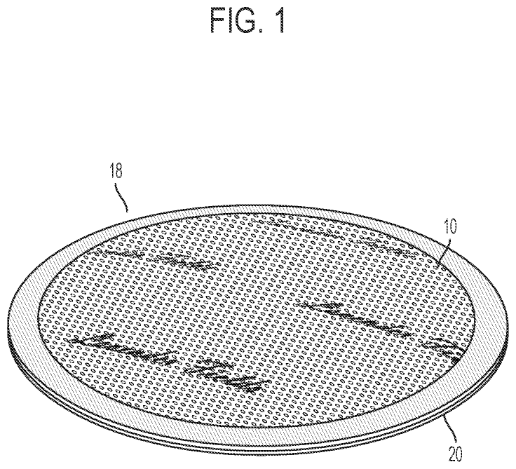

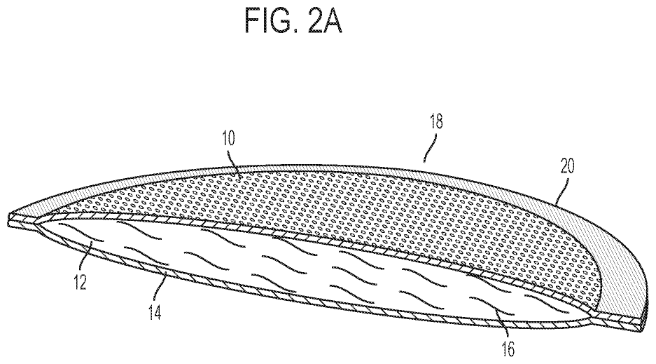

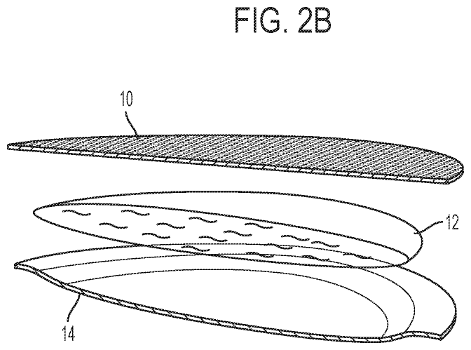

For the purpose of explanation and illustration, and not limitation, an exemplary embodiment of the device is shown in FIGS. 1 and 2A-2B.

The device (18) shown in FIG. 1 has a generally round shape, and convex structure. However, in accordance with one aspect of the invention, the device can be made in a variety of geometric shapes (i.e., square, octagonal, triangular, etc.), for example, to correspond with a specific surface area for desired release rate of the volatile active material and/or for design reasons, such as providing the consumer with a more aesthetically pleasing shape, without departing from the scope of the disclosed subject matter and can furthermore have a variety of shapes when viewed in a side view, including but not limited to substantially planar orientation.

Furthermore, the shape of the device can relate to the fragrance therein, such as the device being in a flower profile shape as corresponding to a floral scent. An upper vapor releasing oleophobic microporous membrane (10) having pores or micropores is shown (not to scale), and is described in more detail below. A perimeter seal (20) is formed by joining the vapor releasing oleophobic microporous membrane with the lower barrier layer (14) along a perimeter of the device shape. The membrane (10) and lower barrier layer (14) together define a cavity therebetween and having a predetermined volume.

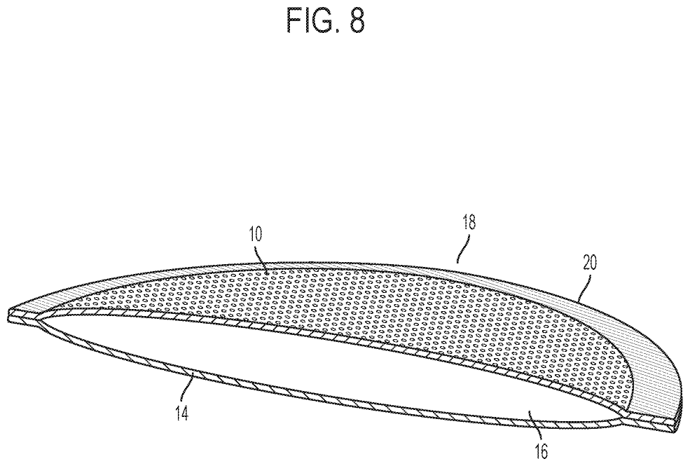

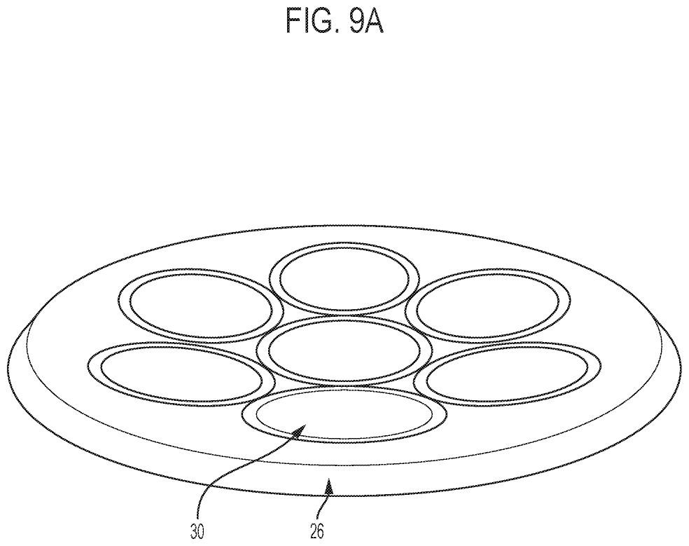

A cross-sectional view of a device (18) is shown in FIG. 2A. The device includes a flexible lower barrier layer (14), the vapor releasing oleophobic membrane (10), the perimeter seal (20), a cavity (16) formed between the lower barrier layer and the vapor releasing membrane, wherein such cavity can be filled with a reservoir substance (12). No head space is required between the lower barrier and the vapor releasing oleophobic membrane, as depicted in FIG. 2A. FIG. 8 depicts a cross-sectional perspective view of the device of FIG. 2A without the reservoir substance (12) therein, according to the disclosed subject matter.

A removable sealing layer (not shown) can further be included with the device of FIG. 2A adjacent the vapor releasing oleophobic membrane (10) and away from the lower barrier layer (14). The removable sealing layer can be selectively removed from the device to expose the vapor releasing membrane to an external environment and enable the release of volatile active material.

In certain embodiments of the disclosed subject matter, the reservoir or cavity of the device can be formed using one or more vapor releasing microporous membranes (10). For example, a perimeter seal can be formed by joining two vapor releasing microporous membranes together along a perimeter of the device shape. The vapor releasing microporous membranes together can define a cavity therebetween having a predetermined volume.

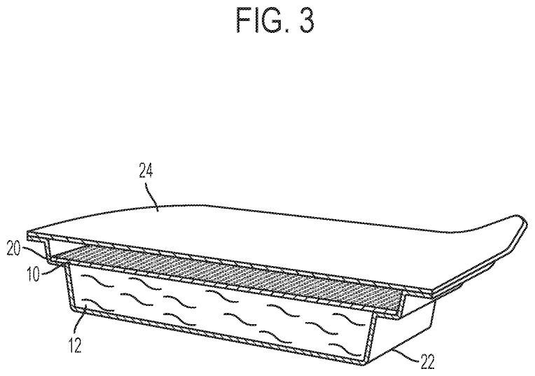

An alternate embodiment of the presently disclosed device is depicted in FIG. 3. In this embodiment, the device includes a lower barrier layer (22) preformed rigid tray, which forms a perimeter seal (20) with the upper vapor releasing membrane (10). An edge/lip of the rigid tray extends beyond the vapor releasing oleophobic membrane to allow for the attachment of an additional removable sealing layer (24). The sealing layer (24) can comprise for example, a heat sealable foil material disposed over the vapor releasing oleophobic membrane to prevent ambient vapor loss during storage. Such sealing layer is removable by a consumer prior to use.

In alternate embodiments, such as where the reservoir or cavity is defined between one or more vapor releasing membranes, a container, such as a tray or other holder can be provided, and the device can be placed in the container. In certain embodiments the container can include a removable sealing layer that can be selectively removed from the device as described above.

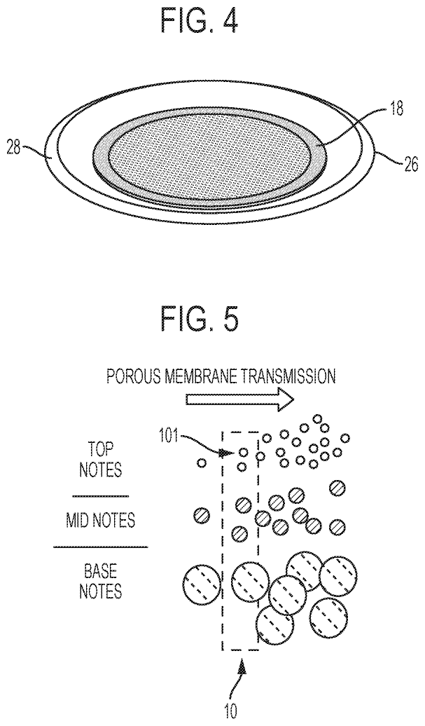

As shown in the system of FIG. 4, the device (18) can be placed in a display unit (26), such as in a dish (28). Alternatively, the device (18) can be placed directly on any suitable surface, such as a desk, vehicle dashboard, shoe racks, and the like. Unlike devices known in the art, such as U.S. Publication No. 2005/0016985, U.S. Publication No. 2015/0283280, U.S. Publication No. 2015/0174278, and U.S. Pat. No. 7,067,772, the contents of each of which are incorporated herein by reference in their entireties, the device according to the disclosed subject matter does not require any heat assistance or energy assistance to release volatile active material therefrom. As such, devices according to the disclosed subject matter are ready-to-use without requiring further accessories, permitting cost savings and safety benefits amongst other benefits. The release of volatile active material, such as a fragrance, from the reservoir substance occurs at ambient temperatures, such as temperatures ranging from approximately 55° F. to approximately 105° F.

The device, once depleted, can be disposed of, by simply transferring the device (18) into a waste receptacle. In contrast, currently available devices using wax melts that are activated by a heat source are not readily disposable. The wax melts in such devices must be physically cleaned out of the warming dishes once they are depleted before a user can introduce another wax melt into the warming unit. Such devices can pose dangers if the heat sources are left on past the depletion of wax melts therein.

### Barrier Layer

In the embodiment shown in FIGS. 1 and 2, the device includes a barrier layer constructed using a flexible, impermeable barrier laminate. In an embodiment of the disclosed subject matter, the barrier laminate comprises a foil layer in a multi-layer construction, which provides an impervious barrier as well as a heat sealable layer of polyethylene or polypropylene. The barrier layer can include any suitable material such as the barrier panel materials as disclosed in U.S. Publication No. 2014/0048614, incorporated herein by reference in its entirety. The external surface of the barrier layer can further include an adhesive to permit the device to stick to a locker door, vehicle dashboard, or the like.

In the embodiment shown in FIG. 3, the barrier layer is formed of a semi-rigid or rigid material, for example but not limitation, polyethylene terephthalate or polyolefin coated aluminum, and is joined to the vapor permeable layer as known in the art, such as, but not limited to, through the use of a mechanical union or through the means of a sonic or heat sealed weld, forming a fluid tight perimeter seal.

### Vapor Permeable Membrane Layer

As embodied herein, the device includes at least one vapor permeable membrane. In certain embodiments, the permeable layer is sealed to a lower barrier layer along the perimeter of the device. In certain embodiments, the two layers are sonically or heat sealed. However, other means of sealing are contemplated herein.

The permeable layer comprises a microporous material having micropores, thereby allowing for the rate of fragrance release into the environment to be controlled via the overall thickness, permeability, porosity, and average pore size of the material. The use of a microporous material enables the presently disclosed device to offer performance advantages, such as a more linear and predetermined rate of release and a prolonged functional life, as compared to currently available wax melts, which result in fully exposed pools of hot molten wax upon being heated. The rate of release of the volatile material, i.e., fragrance, through the permeable layer could be adjusted based on the thickness, permeability, porosity, and average pore size of the microporous materials used. In certain embodiments of the presently disclosed device, the porosity is between about 35% to about 65%, between about 35% to about 60%, between about 35% to 55%, or between about 40% to 55%. In other embodiments of the presently disclosed device, the average pore size is between about 0.01 to 0.07 microns, between about 0.02 to 0.07 microns, or between about 0.03 to 0.07 microns. In further embodiments of the presently disclosed device, the permeable layer has a Gurley permeability rating of about 200 seconds to about 800 seconds.

In some traditional fragrance releasing devices, bleeding, sweating, seepage, or leakages of the fragrance solution through membrane coverings in traditional fragrance releasing devices may occur. Typically, evidence of drops and/or a film of liquid may be visually observed on the surface of the by the naked eye over time. In certain embodiments the microporous membrane material is oleophobic which functions to prevent bleeding, sweating, seepage, or leakages of the volatile material out of the reservoir. The oil repellency of the oleophobic membrane material may be graded using the AATCC Standard Test Method No. 118, which is based on the resistance of a material to penetration of certain oils or mixtures of oils of varying surface tensions using a scale from 1 (i.e., resistant only to the least penetrating of the test oils) to 8 (i.e., resistant to the most penetrating of the test oils) using the test oils shown in the following table:

| Grade | Test Oil |
|---|---|
| 1 | Kaydol |
| 2 | 65/35 Kaydol/n-hexadecane by volume |
| 3 | n-hexadecane |
| 4 | n-tetradecane |
| 5 | n-dodecane |
| 6 | n-decane |
| 7 | n-octane |
| 8 | n-heptane |

A grade of 0 is assigned to materials which are not resistant to the least penetrating of the test oils and not considered oleophobic according to the present disclosure. Higher numbers indicate better oil repellency. In certain embodiments of the presently disclosed device, the microporous material has an oil repellency grade equal to or greater than 3. In some embodiments of the presently disclosed device, the microporous material has an oil repellency grade between 3 and 7. In other embodiments of the presently disclosed device, the microporous material has an oil repellency grade between 4 and 6.

The microporous membrane material may be inherently oleophobic or may be rendered oleophobic by treating the material with an oil resistant coating to achieve the desired oil repellency grade. Thus, in one embodiment, the presently disclosed device comprises a microporous membrane having an oleophobic coating. In one particularly suitable embodiment, a fluorocarbon based coating is applied to the microporous membrane material according to the methods described in U.S. Pat. Nos. 6,270,841 and 7,005,161, incorporated by reference herein, wherein the monomer is selected based on the membrane material to be treated and for oleophobic characteristics. The microporous membrane may be coated on one or both sides of the membrane. For membrane materials treated with an oil resistant coating, it is preferable to use a coating which does not substantially change the permeability and/or porosity of the treated membrane compared to the permeability and/or porosity of the untreated membrane.

In one embodiment of the disclosed subject matter, the rate controlling vapor permeable oleophobic membrane is one of the microporous membranes sold commercially as CELGARD® (Celgard LLC, Charlotte, N.C.) and treated with a flurocarbon based coating as described above. The CELGARD® membrane family is manufactured in a variety of offerings as the permeability, porosity, average pore size, and thickness of the membranes offered vary. Particularly suited to the disclosed subject matter is CELGARD® 4560, which is a composite structure comprising CELGARD® 2500 that is laminated to a loosely structured non-woven polypropylene article. CELGARD® 2500 is a hydrophobic polypropylene in which sub-micron pores are formed in the polypropylene film during processing. CELGARD® 4560 has a base film thickness of 25 μm while the laminated film thickness is approximately 110 μm. The machine direction tensile strength of CELGARD® is 1055 Kg/cm² and the cross direction tensile strength is 135 KG/cm².

In a construction of the presently disclosed device wherein the microporous membrane comprises an oleophobic coating, on only one sides of the membrane, the coating may be situated either on the side of the membrane on the inner side of the cavity in contact with the reservoir substance or on the outer side of the membrane. In a preferred construction of the presently disclosed device, a coated CELGARD® 4560 is orientated with the oleophobic coating on the outer side of the membrane and the non-coated, non-woven side of the membrane on the inner side of the cavity in contact with the reservoir substance/composition, which is then heat sealed to the polyolefin covering on the barrier laminate. For membrane materials treated with an oil resistant coating, the permeability of the treated membrane should be the same or substantially similar to the permeability of the untreated membrane.

In one embodiment, the presently disclosed device comprises a vaper releasing oleophobic microporous layer having a porosity between about 40% and 60%, an average pore size between 0.03 and 0.07 microns, and an oil repellency grade of 4, 5 or 6.

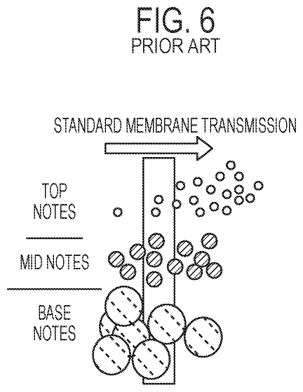

The microporous membrane according to the disclosed subject matter can function as a containment device for the reservoir substance disclosed herein and does not function as a wicking device, as traditional membranes commonly function. The microporous material enables a greater range of olfactive categories for use with the device, in contrast to traditional monolithic membranes, as further discussed with respect to FIG. 5 and FIG. 6.

FIG. 5 depicts a limited cross-sectional view of a vapor releasing oleophobic microporous membrane (10) according to the disclosed subject matter, permitting volatile material therethrough. As depicted, the top, mid, and base notes of the volatile material is permitted through the membrane (10) by direct evaporation through the micropores (101). Although not drawn to scale, in an embodiment wherein the volatile material is contained within a gel matrix (not shown), the micropores (101) are small enough to retain the gel matrix, but large enough to allow fragrance molecules (i.e., notes) to evaporate therethrough enabling less restrictions on the evaporation of volatile material. Therefore, the range of olfactive categories applicable to the presently disclosed subject matter is greater than that of traditional devices such that fragrance types having heavier aroma ingredients (i.e., lower vapor pressure notes) are usable with the device.

In contrast, and as depicted by the prior art traditional monolithic membrane of FIG. 6, the traditional membrane is limited with respect to the kinds of fragrances that can be used and to the intensity of the fragrance. Traditional membranes have been limited to liquid formulations with larger fragrance molecules that are unable to diffuse through the traditional membranes. As depicted, a limited number of base notes and mid notes are permitted to dissipate through the traditional monolithic membrane. The fragrance molecules must first diffuse through the traditional membrane prior to evaporation. As such, the olfactive range of fragrances for traditional membranes is limited.

It is possible that by selecting a membrane with a different permeability and/or porosity and/or average pore size and/or thickness and/or oil repellency grade that the opportunity exists for using the membrane as a rate controlling mechanism in exerting influence over the rate of release of the active agents while preventing leakage, seepage, or bleeding of the volatile material out of the reservoir. This form of control is especially important if the currently disclosed device is used for the dispersion of insecticides and medicants or medicaments.

Another unique aspect which pertains to the use of a microporous rate controlling membrane is the ability to print on the membrane with text, designs, logos and the like and have the colors and designs remain stable when in contact with the contents of the reservoir and heat exposure. The aesthetic contribution offered by this print option especially in support of a fragrance positioning makes the currently disclosed cartridge far superior to the wax melts which can only be differentiated by color. The ability to print the fragrance name or a picture of a scene on the cartridge in support of fragrance positioning is a valuable visual contribution offered by present device. Although the printing can block the microporous openings in the membrane depending on the material used for printing, a lightly designed pattern or design will not cause any significant reduction in the ability of the cartridge to release its active agents.

In certain embodiments, a polyolefin based material is utilized for its heat sealing capabilities, thereby allowing for an easily sealed perimeter during manufacture of the device of the disclosed subject matter. Non-limiting examples of microporous polyolefin based materials suitable for use in the present device once treated with an oleophobic coating include, but are not limited to filled porous polyethylene films available under the tradename Teslin® (PPG Industries, Pittsburgh, Pa.) and Tyvek® (E.I. du Pont de Nemours and Company, Wilmington, Del.), among other suitable manufacturers. Teslin® membranes, in particular, are compatible with a broad range of print processing and can be utilized in embodiments of the present device intended to have a printed design. Other suitable membranes include those as commercially sold by Bluetek.

### Reservoir Substance

The cavity or reservoir of the device can be filled with a reservoir substance comprising a volatile material (i.e., fragrance or fragrance composition). The cavity includes a predetermined volume. The volatile composition can be selected from a variety of options to include fragrances, aroma therapeutic compositions, medicants, medicaments, decongestants, insect repellants, insecticides and the like. In some embodiments the volatile material can be dispersed in a matrix material

### Volatile Materials

As disclosed herein, volatile materials can include different fragrances or fragrance compositions. In certain embodiments, the fragrances can include, but are not limited to, floral fragrances, musky fragrances, wood fragrances, and combinations thereof. The fragrance types are formulated as a single chemical compound or as fragrance compositions comprising one or more chemical compounds. Additionally, such fragrances or fragrance compositions can be used at higher levels than was is used in a traditional membrane.

In a particular embodiment, the fragrance can be a floral fragrance, such as lily of the valley. Non-limiting examples of floral fragrance include Lilial®, Kovanol®, Florol®, Heliobouquet®, and combinations thereof. Lilial® can be butylphenyl methylpropional. Kovanol® can be 4-(4-Hydroxy-4-methylpentyl)-1-cyclohex-3-enecarboxaldehyde). Florol® can be floral pyranol or 4-methyl-2-(2-methylpropyl)oxan-4-ol. Heliobouquet® can be ocean propanal or 3-(1,3-benzodioxol-5-yl)-2-methylpropanal.

In another embodiment, the fragrance can be a musky fragrance. In a particular embodiment, the musky fragrance can be galaxolide, ethylene brassilate (Musk T®), and combinations thereof. Musky fragrance materials can be used in amounts of from about 1% to about 2% up to from about 20% to about 30%, based on the total amount of the reservoir substance.

In another embodiment, the fragrance can be a woody fragrance. In a particular embodiment, the woody fragrance can be Iso E Super®, Vertenex®, and combinations thereof. Iso E Super® can include tetramethyl acetyloctahydronaphthalene. Vertenex can include 4-tert-butylcyclohexyl acetate. Woody fragrance materials can be used in amounts of up to about 40%, based on the total amount of the reservoir substance.

Traditional membranes, as described with respect to FIG. 6, must utilize one or more carriers, diluents or surfactants in their fragrance compositions containing heavier note fragrances to help drive and carry materials, such as some woody fragrances, through a traditional membrane. Those carriers, diluents, or surfactants act as a thinner or thinning agent for heavier note fragrances and require a certain volume percentage of the fragrance components to enable such traditional membranes to perform, as desired. Unfortunately, the volume dedicated to these additional ingredients for traditional membranes would otherwise replace supplemental volume available for the fragrance itself, causing a lower percentage of fragrance being used, which is undesirable.

In contrast, with the embodiments of the disclosed subject matter, carriers, diluents, and surfactants are not required to deliver the fragrance or fragrance compositions through the microporous membrane, as these microporous membranes are inherently sufficient to deliver fragrance without the use of a carrier or diluent. Thus, the reservoir composition according to the disclosed subject matter can contain a higher percentage of fragrance not otherwise available in traditional device, and the intensity of such fragrances can be increased as compared to such traditional devices.

### Matrix Material

In addition to the volatile materials, in certain embodiments, the reservoir substance may also contain a matrix material for delivering fragrance to an external environment via the microporous membrane.

The cavity of the present device is accepting of a wide variety of reservoir forms, including solids, semi-solids, viscous liquids, non-thickened fragrance oil. Non-limiting examples of reservoir options include, but are not limited to paraffin waxes, soy waxes, wax blends, wax and oil blends, metallic soap based gels, elastomeric gels, gels formed using modified clays, e.g., bentonite gels or colloidal silica gels. In a particular embodiment, the reservoir substance is not a water-based solution, rather it is a gel-based, thereby allowing the reservoir substance to contain a high percentage of fragrance and produce a fragrance of high intensity. The final consistency of the reservoir composition is highly impacted by the amount of volatile materials dispersed within the matrix of the reservoir and the rheological format of the reservoir substance should be such that at the consistency is suitable for operation at ambient temperatures.

In specific embodiments, the matrix material is a gel material. Importantly, the gel material is not a liquid. The gel material contains the volatile material therein to further prevent any bleed, sweating, leakages or spilling of the volatile material. Such embodiments of the presently disclosed device are particularly suitable for uses wherein the orientation of the device may change or spilling of the volatile material is undesirable, such as use of the device to provide fragrance in a gym bag.

In certain embodiments, the viscosity of the reservoir substance includes a viscosity dimension, which can range from about 500 cps to about 8500 cps at ambient temperature. The reservoir substance accordingly does not include fillers that traditional systems typically require. However, the reservoir substance can comprise a high heat resistant olefin combined with an elastomeric polymer thickener and volatile materials to ensure controlled fragrance release within the viscosity range to keep the reservoir substance intact under the microporous membrane to avoid spills, leakage, or the like. Traditional devices that utilize gel substances without a membrane require the gel substance to consist of a rubberized solid outside the viscosity ranges disclosed herein. Such materials hinder diffusion of suitable materials according to the disclosed subject matter.

Conventional wax melts and wax tarts must be sufficiently solid in order to retain their shape. For this reason, conventional fragrance loadings are kept low, with a range of about 6% to about 12%, by weight, being common, and with an inability to exceed 12% maximum by weight. As the fragrance medium of the present device is not limited to a solid shaped composition, fragrance loadings as high as about 100% can be achieved. In particular fragrance loadings of between about 30%-100%, between about 30%-99%, between about 30%-90%, between about 40%-80%, between about 45%-75% can be achieved. The reservoir rheology along with the fragrance loading must be balanced in such a way that the fluid nature of the reservoir composition at ambient temperatures does not permeate the micropores of the vapor permeable layer and result in a fluid accumulation on the outside of the device.

### Use of the Device

The disclosed subject matter further contemplates a method of using the device. When the device is provided with a removable sealing layer, the sealing layer is removed and the device is subsequently in a ready-to-use condition permitting fragrance notes to dissipate through the membrane. The sizing of the device can vary, including smaller or reduced formats or larger units that are stand alone or capable of segmenting into multiple pieces.

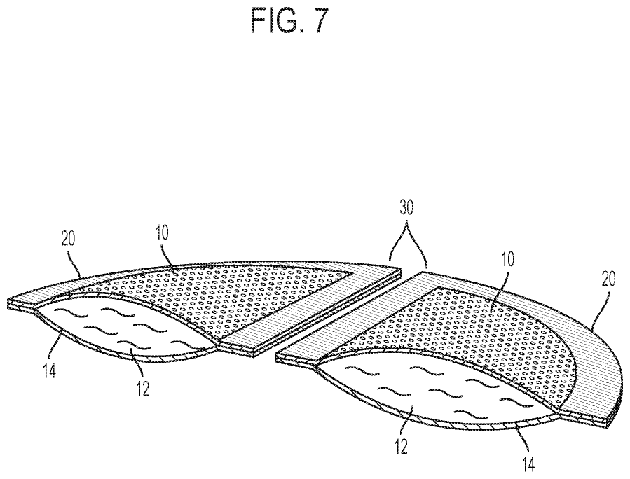

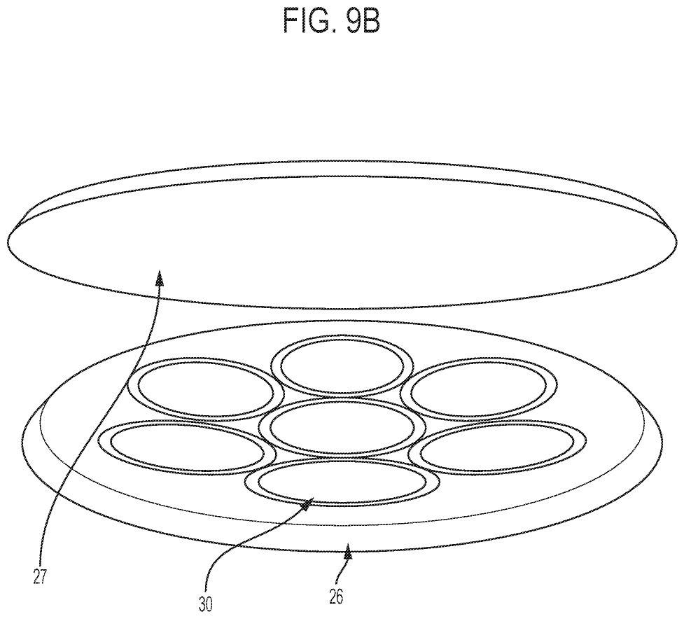

In one embodiment, the device can be formed in a reduced size format such that a plurality of devices, such as two devices (30), can be use simultaneously, as shown in the system of FIG. 7. In this way, the user can create fragrance combinations of their own choosing giving them creative control over the fragrance experience they desire. The reduced size format can also be better suited for smaller areas, such as a locker or shoe box. Such an option is not available with traditional devices. FIG. 9A depicts an example of reduced size devices (30) on a display unit (26), according to the disclosed subject matter. Such reduced size devices can also allow a user to vary the intensity of the device to fit the user's desires.

With smaller units or a larger unit segmented into multiple pieces, the consumer can choose how many units they would like to activate based on individual preference and the size of the surrounding environment. For example, a smaller space such as a closest, bathroom, or locker can only require a single device, whereas multiple devices can be better suited for a larger open floorplan. The compact convenient size allows the user to customize their experience such that the smaller devices can be more versatile. FIG. 9B depicts a reduced size device on a display unit (26) with a containment lid (27) according to the disclosed subject matter. The lid can enclose the tray to retain the fragrance therein to allow the use to utilize the devices at a personalized time.

In certain embodiments the device can be suspended. For example, the barrier layer or container or other holder can contain an aperture therein disposed in an area that does not interfere with either a seal between the vapor permeable membrane and the barrier layer or a seal between the container and a sealing layer. For example, the aperture can be disposed in a non-sealing area of a flange of the barrier layer or container. Using the aperture, the unit can be suspended, e.g., hung from a hook, either directly or by looping a string or the like through the aperture. Such embodiments can be used, for example, as a car air freshener.

### EXAMPLES

The presently disclosed subject matter will be better understood by reference to the following Example, which is provided as exemplary of the disclosure, and not by way of limitation.

### Example 1

Two versions of the presently disclosed devices having a foil barrier layer and a vapor releasing oleophobic microporous layer comprising a microporous membrane treated with an oleophobic coating were prepared. In both devices the same microporous membrane was used. Device A comprised a microporous layer with an oil repellency grade of 4 and Device B comprised a microporous layer with an oil repellency grade of 5. The devices contained 100% of neat fragrance oil in the reservoir (8 gram fill). The devices were placed in an oven at 45° C. and 75% relative humidity. The devices were inspected after one week for leakage through the barrier layer or the microporous layer by placing each side of the device down onto a mirror and checking for oil smudge. No leakage through the barrier layer or the microporous layer was observed on either device. The devices were inspected again after two weeks using the same methodology. Slight oil was detected on the surface of the microporous layer of Device A. No oil was detected on the surface of the microporous layer of Device B.

Although the presently disclosed subject matter and its advantages have been described in detail, it should be understood that various changes, substitutions and alterations can be made herein without departing from the spirit and scope of the application as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Patents, patent applications publications product descriptions, and protocols are cited throughout this application the disclosures of which are incorporated herein by reference in their entireties for all purposes.

What is claimed is:

1. A fragrance delivery device, comprising:
a vapor releasing microporous layer having a plurality of micropores therein;
a flexible impermeable barrier layer coupled to the vapor releasing microporous layer at a perimeter thereof, wherein the microporous layer and barrier layer define a cavity; and
a reservoir substance disposed in the cavity, wherein a volatile material of the reservoir substance is releasable from the fragrance delivery device via the plurality of micropores when the fragrance delivery device is exposed to an ambient temperature.

2. The fragrance delivery device of claim 1, wherein the vapor releasing microporous layer is a vapor releasing oleophobic microporous layer.

3. The fragrance delivery device according to claim 2 wherein the microporous layer has an oil repellency grade equal to or greater than 4.

4. The fragrance delivery device of claim 2, wherein the vapor releasing oleophobic microporous layer has a porosity between about 40% and 60%, an average pore size between 0.03 and 0.07 microns, and an oil repellency grade of 4, 5 or 6.

5. The fragrance delivery device according to claim 1, wherein the reservoir substance comprises a volatile material and a matrix material, and wherein the reservoir substance has a viscosity ranging from about 500 cps to about 8500 cps at ambient temperature.

6. A method of using a fragrance delivery device, comprising:
providing a fragrance delivery device according to claim 1 having a removable sealing layer;
removing the sealing layer from the fragrance delivery device; and
exposing the fragrance delivery device to an external environment.

7. A method of manufacturing a fragrance delivery device, comprising:
providing a vapor releasing microporous layer;
coupling a flexible impermeable barrier layer to the vapor releasing microporous layer, wherein the microporous layer and barrier layer define a cavity; and
depositing a reservoir substance into the cavity, wherein a volatile material of the reservoir substance is releasable from the fragrance delivery device via micropores of the microporous layer when the fragrance delivery device is at ambient temperature; and
sealing the vapor releasing microporous layer with the flexible impermeable barrier layer at a perimeter thereof.

8. The method of claim 7, wherein the vapor releasing microporous layer is a vapor releasing oleophobic microporous layer.
